SEQUENCE LISTING (5 sequences)

SEQ ID NO: 1 - protein, 138 aa, Homo sapiens
PLTLKPIRPRKYPNRPSKTPLHERPHACPAEGCDRRFSRSDELTRHLRIHTGHKPFQCRICMRSFSRSDHLTTHIRTHTGEKPFACEFCGRKFARSDERKRHAKIHLKQKEKKAEKGGAPSASSAPPVSLAPVVTTCA

SEQ ID NO: 2 - protein, 138 aa, Mus musculus
PLTLKPIRPRKYPNRPSKTPLHERPHACPAEGCDRRFSRSDELTRHLRIHTGHKPFQCRICMRSFSRSDHLTTHIRTHTGEKPFACEFCGRKFARSDERKRHAKIHLKQKEKKSEKGGAPSASSAPTVSLAPVVTTCA

SEQ ID NO: 3 - protein, 387 aa, mus musculus
MTGKLAEKLPVTMSSLLNQLPDNLYPEEIPSALNLFSGSSDSVAHYNQMATENVMDIGLTNEKPNPELSYSSSFQPAPGNKTVTYLGKFAFDSPSNWCQDNIISLMSAGILGVPPASGALSTQTSTASMVQPPQGDVEAMYPALPPYSNCGDLYSEPVSFHDPQGNPGLAYSPQDYQSAKPALDSNLFPMIPDYNLYHHPNDMGSIPEHKPFQGMDPIRVNPPPITPLETIKAFKDKQIHPGFGSLPQPPLTLKPIRPRKYPNRPSKTPLHERPHACPAEGCDRRFSRSDELTRHLRIHTGHKPFQCRICMRSFSRSDHLTTHIRTHTGEKPFACEFCGRKFARSDERKRHAKIHLKQKEKKSEKGGAPSASSAPTVSLAPVVTTCA

SEQ ID NO: 4 - protein, 387 aa, homo sapiens [sequence omitted]

SEQ ID NO: 5 - protein, 173 aa, homo sapiens
MDPIRVNPPPTTPLETIKAFKDKQIHPGFGSLPQPPLTLKPIRPRKYPNRPSKTPLHERPHACPAEGCDRRFSRSDELTRHLRIHTGHKPFQCRICMRSFSRSDHLTTHIRTHTGEKPFACEFCGRKFARSDERKRHAKIHLKQKEKKAEKGGAPSASSAPPVSLAPVVTTCA

(19) United States
(12) Patent Application Publication
(10) Pub. No.: US 2012/0253013 A1
(43) Pub. Date: Oct. 4, 2012

(54) Early Growth Transcription Factors and Their Therapeutic Use

(76) Inventors: Dan Liu, London (GB); Ian Charles Zachary, London (GB)

(21) Appl. No.: 13/391,681

(22) PCT Filed: Aug. 25, 2010
(86) PCT No.: PCT/GB2010/051410

§ 371 Date: Apr. 23, 2012

(30) Foreign Application Priority Data
Aug. 25, 2009 (GB) 0914855.2

(51) Int. Cl. C07K 14/47; C12N 15/12; C12N 15/861

(52) U.S. Cl. 530/350; 435/320.1; 536/23.5

(57) ABSTRACT

The present invention comprises a nucleotide which encodes a protein, wherein the protein includes an amino acid sequence having at least 95% homology to SEQ ID NO: 1 (Egr3 DNA binding domain), for use in therapy.

### FIELD OF THE INVENTION

The present invention relates to Egr transcription factors, and their use in therapy.

### BACKGROUND OF THE INVENTION

Egr3 is a member of the zinc finger Egr transcription factor subfamily, which is upregulated by vascular endothelial growth factor (VEGF). Inhibition of Egr3 gene expression by RNA interference suppresses endothelial proliferation, migration and tubulogenesis.

The Early Growth Response (Egr) subfamily of zinc finger transcription factors were first discovered as genes upregulated in response to growth factors and involved in regulation of cellular growth and differentiation. This subfamily comprises four members, Egr1, Egr2, Egr3 and Egr4, each of which contains a highly conserved zinc finger DNA binding domain recognising an identical nine-base-pair segment of DNA, the Egr response element (ERE). Once Egrs bind to the promoter of target genes, they regulate the expression of target genes to participate cellular functions. However little is known about the mechanisms mediating Egr-regulated cellular functions.

Egr3 is implicated in nerve growth and differentiation and in nerve growth factor (NGF) mediated cell signalling. Egr3 is the most strongly upregulated gene by nerve growth factor (NGF) in pheochromcytoma 12 (PC12) cells and overexpression of a dominant negative Egr3 inhibited NGF induced neurite outgrowth in PC12 cells. Egr3-deficient mice displayed gait ataxia due to lack of development of muscle spindles, skeletal muscle sensory organs which consist of encapsulated muscle fibres innervated by motor or sensory axons, indicating that Egr3 plays a pivotal role in regulation of sensory axon and myotube interaction during muscle spindle morphogenesis. A recent study also demonstrated that Egr3-deficient mice display abnormal sympathetic nervous system development and exhibited physiological dysautonomia, indicating that Egr3 is an NGF signalling effector that regulates sympathetic neuron gene expression for normal target tissue innervation and function. Furthermore, Egr3 is downregulated in motor neurons in sporadic amyotrophic lateral sclerosis, a motor neuron disorder.

Egr3 also regulates thymocyte proliferation and survival. In Egr3-deficient mice there was a reduction of number of thymocytes in comparison with wild type mice as a result of poor proliferation. However, transgenic mice that constitutively express Egr3 also have reduced thymic cellularity due to poor survival with reduced expression of Bcl-XL.

Interestingly, a recent study revealed that Egr3 is an estrogen-induced gene in breast cancer cells. Overexpression of Egr3 in the breast cancer cell line MCF7 identified NAB2 as one of the target genes for Egr3. Moreover, Egr3 immunoactivity was detected in more than half of breast carcinoma tissues and was positively associated with lymph node status, distant metastasis and estrogen receptor expression. Egr3 expressing transformed breast cancer cells enhanced migration and invasion property.

While the role of Egr3 in neuronal cells and lymphocytes is relatively well characterised, the functions of Egr3 in vasculature is not well understood. A previous study reported that Egr3 was transiently and strongly expressed after VEGF treatment in human umbilical vein endothelial cells (HUVECs). It has also been demonstrated that Egr3 knockdown inhibited several important VEGF mediated functions including proliferation, migration and tubulogenesis by RNA interference. However the mechanisms by which Egr3 mediates endothelial functions are unclear.

U.S. Pat. No. 5,837,692 discloses the use of viral vector—delivered Egr-1 for inhibiting the growth of a tumour. The experimental data were obtained using a mouse fibroblast model, rather than in human tumour cell lines. Mouse fibroblast model is not a reliable indicator of actual therapeutic effect in humans.

### SUMMARY OF THE INVENTION

Two adenoviral vectors were generated; one encoding a full-length human Egr3 (Ad-Egr3) and one encoding a N-terminal truncated Egr3 with a deletion of both transactivation domains (Ad-ΔEgr3). Overexpression of Egr3 increased its binding to the Egr response element and induced the known Egr3 target gene NAB2 expression. The wild type Ad-Egr3 increased endothelial migration, whereas Ad-ΔEgr3 infected cells increased in Egr DNA binding, but failed to induce NAB2 gene expression and cell migration. Furthermore conditioned medium from Ad-Egr3-infected cells stimulated uninfected HUVEC cell migration, indicating that Egr3 induced production of chemoattractants. Affymetrix oligonucleotide microarray screening of Ad-Egr3-infected cells identified several upregulated genes involved in cell migration including hepatocyte growth factor (HGF). Moreover, conditioned medium from Ad-Egr3 infected cells was able to activate HGF receptor c-Met phosphorylation. Knockdown of HGF mRNA prior to Ad-Egr3 infection reduced Ad-Egr3 induced HGF production and cell migration. In addition, Ad-Egr3 infected cells changed morphology from cobblestone shape to spindle shape. This study identified a novel downstream mechanism for Egr3 mediated cell migration of endothelial cells involving Egr3-dependent autocrine production of HGF and activation of c-Met receptor.

A number of further downstream effects were observed in this study, which allowed for the prediction, and subsequent testing of a number of conditions where Egr3 and ΔEgr3 are useful.

According to a first aspect, the present invention is a nucleotide which encodes a protein, wherein the protein includes an amino acid sequence having at least 95% homology to SEQ ID NO: 1 (Egr3 DNA binding domain), for use in therapy.

According to a second aspect, the present invention is a vector comprising a nucleotide as defined above, for use in therapy.

According to a third aspect, the present invention is a protein as defined above, for use in therapy

### DESCRIPTION OF THE SEQUENCE LISTING

SEQ ID NO: 1 is human Egr3 DNA binding domain (DBD).

SEQ ID NO: 2 is murine Egr3 DNA binding domain (DBD)

SEQ ID NO: 3 is murine Egr3.

SEQ ID NO: 4 is human Egr3

SEQ ID NO: 5 is human ΔEgr3.

### DESCRIPTION OF PREFERRED EMBODIMENTS

The present invention is a nucleotide which encodes a protein, wherein the protein includes an amino acid sequence having at least 95% homology to SEQ ID NO: 1 (Egr3 DNA binding domain), for use in therapy.

Preferably, the homology is 96%, 97%, 98% or 99%. More preferably, the homology is 100%. In other words, a nucleotide of the invention encodes a protein, wherein the protein includes the Egr3 DNA binding domain, or a mutant thereof having substantially the same activity, for use in therapy.

A number of downstream effects of Egr3 have been discovered. It has been discovered that over-expression of Egr3 promotes HGF production. This has potential application in haematopoetic stem cell differentation, as HGF promotes differentiation and proliferation of haematopoetic stem cells.

Over-expression of Egr3 also increases expression of CD52 (the antigen for Campath-1 antibody). Therefore, ΔEgr3 (or the Egr3 DBD), which decreases Egr3 expression, may be useful in the treatment, of cancer of acute promyelocytic leukemia. Egr3 DBD or ΔEgr3 may also be useful for inhibiting malignant in particular melanoma invasion and metastasis.

An increase in expression of APOE has also been observed when Egr3 is over-expressed, meaning that Egr3 could have benefit in the treatment of atherosclerosis and Alzheimer's disease.

Other conditions in which Egr3 could have utility are diseases where neuronal regeneration is beneficial. Therefore, preferably the therapy is of peripheral neuropathy, nerve injury, spinal injury or stroke. A decrease of Egr3 could be beneficial in neovascular conditions. Therefore, preferably the therapy is of cancer, diabetic, retinopathy, atherosclerosis or rheumatoid arthritis. Nucleotides of the invention which block the normal effects of Egr3 an gene transcription are ΔEgr3 and Egr3 DBD.

The present invention includes active fragments or mutants of the nucleotides of the invention, i.e. fragments or mutants of the Egr3 nucleotide, which have substantially the same biological activity at the Egr3 receptor as the nucleotides of the invention. Fragments of interest are, say at least 15 amino acids long, e.g. up to 40 or more amino acids.

DNA sequences for use in the invention may, for example, be genomic DNAs or cDNAs, or hybrids between genomic DNA and cDNA, or they may be synthetic or semi-synthetic. They may originate from any species, though DNAs encoding human nucleotides of the invention are preferred. They may be single-stranded or double-stranded.

DNA sequences for use in the invention may differ from the sequence of the invention by the deletion, insertion or substitution of one or more nucleotides, provided that they encode a protein having Egr3 activity. Similarly, they may be truncated or extended by one or more nucleotides provided that they encode a protein having Egr3 activity.

RNA sequences are also suitable for the practice of the invention. The invention also provides for the use of RNA sequences that are related to these sequences in any of the ways described above for DNA sequences. RNA sequences for the invention may be single-stranded or double-stranded. RNAs of the invention may be of any origin. For example, they may originate from any species, although RNAs encoding human nucleotides are preferred. Synthetic DNAs may also be used, as may semi-synthetic RNAs. Further, DNA transcribed form bacterial plasmids in vivo or in vitro may be used.

It will be appreciated by those of skill in the art that, in RNA sequences suitable for the practice of the invention, the T residues will be replaced by U.

The present invention also includes proteins having at least 95% homology to, and preferably 100% to SEQ ID NO: 1, for use in therapy. The proteins or nucleic acids of the invention are preferably delivered in the form of a pharmaceutical formulation comprising a pharmaceutically acceptable carrier. Any suitable pharmaceutical formulation may be used.

In another preferred embodiment, a viral vector comprises a nucleotide sequence encoding a protein, as defined above. The viral vector may be any vector suitable for delivering the nucleotide to the open wound. The viral vector may be a lentiviral vector, a baculovirus or an adenovirus.

Viral vectors of the invention are preferably disabled, e.g. replication-deficient. That is, they lack one or more functional genes required for their replication, which prevents their uncontrolled proliferation in vivo and avoids undesirable side-effects of viral infection. Preferably, all of the viral genome is removed except for the minimum genomic elements required to package the viral genome incorporating the Egr3 nucleic acid into the viral coat or capsid. For example, it is desirable to delete all the viral genome except the Long Terminal Repeats (LTRs) and a packaging signal. In the case of adenoviruses, deletions are typically made in the E1 region and optionally in one or more of the E2, E3 and/or E4 regions.

Viruses of the invention may be disabled by any suitable technique. For example, genomic deletions may involve complete removal of genes required for replication, or only partial removal. Complete removal is preferred. In general, preferred deletions are of genes required for early transcription of viral genes.

Replication-competent self-limiting or self-destructing viral vectors may also be used.

In general, the nucleic acid for use in the invention will be comprised within an expression construct that ensures their expression in vivo after they have been delivered to the artery, preferably by a vector as defined above. Such constructs typically comprise a promoter capable of directing the expression of the Egr3 nucleic acid (and optionally a regulator of the promoter), a translational start codon and, operably linked to the promoter, the nucleic acid of the invention. Preferably, these components are arranged in a 5′-3′ orientation.

The construct may also comprise any other suitable components. For example, the construct may comprise a nucleic acid encoding a signal sequence, so positioned in such a position relative to the nucleic acid of the invention that, when it is translated, it is capable of directing the expressed Egr3 protein to a given cell type or cell compartment. Any such signal sequence will typically be positioned immediately 3′ or immediately 5′ to the Egr3 nucleic acid, such that the signal sequence and Egr3 protein are translated as a single fusion protein, with the signal sequence at the C- or N-terminus.

The construct may also comprise an enhancer which enhances the degree of expression provided by the promoter. Any enhancer which enhances the expression provided by the selected promoter may be used. For example, in the case of the CMV early gene promoter, the CMV early gene enhancer may be used.

Optionally, the construct may comprise a transcriptional terminator 3′ to the nucleic acid of the invention. Any suitable terminator may be used.

Optionally, the construct may comprise a polyadenylation signal operably linked 3′ to the nucleic acid of the invention.

Optionally, the construct may comprise one or more selectable marker genes, e.g. of antibiotic-resistance, to allow selection of transformed cells in culture. For example, cells may be selected for antibiotic resistance in order.

Optionally, the construct may comprise one or more introns, or other non-coding sequences, for example 3′ or 5′ to the Egr3 nucleic acid.

Any suitable promoter may be used to control the expression of the nucleic acid of the invention. In general, it is preferred to use a viral promoter or a promoter adapted to function in the species of the subject to be treated. Thus, in the case of a human subject, it is preferred to use viral promoters, especially promoters derived from viruses that infect humans, or promoters derived from human genes. Optionally, a promoter may be used in combination with any suitable enhancer.

Desirably, a “strong” promoter is used, i.e. one that secures high levels of expression of the Egr3 protein of the invention. Promoters that achieve overexpression of the Egr3 protein are desirable. Preferred promoters include the cytomegalovirus (CMV) promoter, optionally in combination with the CMV enhancer; the human β-actin promoter; the simian virus 40 (SV40) early gene promoter; the Rous sarcoma virus (RSV) promoter; and the retroviral long terminal repeat (LTR) promoter.

Promoters, and other construct components, are operably linked to the Egr3 nucleic acid. Thus, they are positioned in order that they may exert their effect on expression of the Egr3 nucleic acid. For example, in the case of a promoter, the promoter is positioned relative to the Egr3 nucleic acid such that it is able to direct expression of the Egr3 nucleic acid. Desirably, construct components are positioned to allow them to exert their maximum effect on expression.

The nucleic acids or constructs use in the invention, may be incorporated into viral genomes by any suitable means known in the art. Viral genomes may then be packaged into viral coats or capsids by any suitable procedure. In particular, any suitable packaging cell line may be used to generate viral vectors of the invention. These packaging lines complement the replication-deficient viral genomes of the invention, as they include, typically incorporated into their genomes, the genes which have been deleted from the replication-deficient genome. Thus, the use of packaging lines allows viral vectors of the invention to be generated in culture.

The proteins, nucleic acids and vectors may be delivered in any suitable dosage, and using any suitable dosage regime. Those of skill in the art will appreciate that the dosage amount and regime may be adapted to ensure optimal treatment of the particular condition to be treated, depending on numerous factors. Some such factors may be the age, sex and clinical condition of the subject to be treated.

For example, viral vectors may be delivered in doses of from 10⁴ to 10¹⁴ cfu or pfu/ml, for example 10⁴ to 10⁶, 10⁶ to 10⁸, 10⁸ to 10¹⁰, 10¹⁰ to 10¹² or 10¹² to 10¹⁴ cfu or pfu/ml. Doses in the region of 10⁵ to 10⁹ cfu or pfu/ml are preferred. The term pfu (plaque-forming unit) applies to certain viruses, including adenoviruses, and corresponds to the infectivity of a virus solution, and is determined by infection of an appropriate cell culture, and measurement, generally after 48 hours, of the number of plaques of infected cells. The term cfu (colony-forming unit) applies to other viruses, including retroviruses, and is determined by means known in the art generally following 14 days incubation with a selectable marker. The techniques for determining the cfu or pfu titre of a viral solution are well known in the art.

For retroviruses, dosages in the region of 10⁵ to 10⁶ cfu/ml are particularly preferred. For pseudotyped retroviruses, dosages in the region of 107 cfu/ml are particularly preferred. For adenoviruses, dosages in the region of 109 pfu/ml are particularly preferred.

The following study illustrates the invention.

In this study, wild type Egr3 (Ad-Egr3) and a truncated Egr3 mutant (ΔEgr3) were over expressed in HUVECs using adenoviral vectors. Ad-Egr3 expressed cells were highly motile in comparison with Ad-ΔEgr3-, Ad-lacZ-infected cells and uninfected cells, and also increased in secretion of HGF and other chemokines. Inhibition of HGF expression by RNA interference prior to Ad-Egr3 infection suppressed Egr3 mediated HGF expression and attenuated chemotaxis on uninfected endothelial cells. This demonstrates a novel mechanism of Egr3 mediated cell migration, involving autocrine HGF production and c-Met phosphorylation, a process important for angiogenesis in embryonic development and in diseases in adult life.

### Materials and Methods

Human recombinant CCL25, CX₃CL1, CCL1, CXCL14, HGF, and VEGF were purchased from R&D Systems.

### Generation of Recombinant Adenoviral Egr3 and ΔEgr3 Constructs

Adenoviral Egr3 and N-terminal truncated Egr3 were generated using the Gateway System (Invitrogen) according to the manufacturer's instructions. Briefly, a double stranded DNA encoding the full length open reading frame of Egr3 or encoding Egr3 with a deletion of the first 214 amino acids of the open reading frame were chemically synthesised and cloned into a pENTR vector (Invitrogen) by GeneScript, USA. The LR recombination of pENTR-Egr3 and pENTR-ΔEgr3 into the destination vector pAd/CMVN5-DEST (Invitrogen) was carried out using the LR Clonase II mix according to manufacturer's instructions (Invitrogen). The pAd/CMVN5-Egr3 and pAd/CMVN5-ΔEgr3 were then transformed and amplified in bacteria TOP10 (Invitrogen). The purified pAd/CMVN5-Egr3 and pAd/CMVN5-ΔEgr3 were digested with Pad to expose the viral inverted terminal repeats before transfection into adenoviral producer the cell line 293A. The final adenoviral Egr3 (Ad-Egr3) and N-terminal truncated Egr3 (Ad-ΔEgr3) were amplified and purified from 293A cells and titrated by plaque assay. The control adenovirus pAd/CMVN5/lacZ was purchased from Invitrogen and amplified, purified and titrated in parallel.

### Cell Culture and Adenoviral Infection and Collection of Conditioned Medium

Human umbilical vein endothelial cells (HUVECs) were purchased from TCS CellWorks, Buckinghamshire, UK. Cells were cultured in endothelial basal medium (EBM) supplemented with 10% foetal bovine serum (FBS), 10 ng/ml human epidermal growth factor (hEGF), 12 μg/ml bovine brain extract, 50 μg/ml gentamicin sulphate and 50 ng/ml amphotericin-B (Cambex, UK). Infection of adenoviruses in subconfluent HUVECs was carried out by addition of virus at MOI 100 into growth medium and incubation with cells for the indicated experimental periods.

Twenty four hours before collection of conditioned medium, cells were transferred to serum free (SF) EBM. Supernatant conditioned medium were concentrated by centrifugation at 4000 rpm in Amicon Ultra Centrifugal Filter Devices (Millipore, Ireland) at 4° C. for 40 min. The resulting conditioned medium was either stored at −20° C. or applied to ELISAs or cell migration assays immediately.

### Small Interfering (si) RNA Transfection

For transfections, chemically synthesized duplex siRNAs directed against human HGF (J-006650-05) and a non-targeted negative control siRNA were purchase from Dharmacon (Germany). 200 nM siRNA was diluted with OptiMEM (Invitrogen) in a final volume of 1440 μl. 80 μl of Oligofectamine (Invitrogen) and 80 μl of OptiMEM I (Invitrogen) were pre-mixed in a separate tube. Both mixtures were allowed to incubate at room temperature for 10 min, and then combined and incubated for 25 minutes at room temperature. Confluent cells in T75 flasks were washed with OpitMEM once and transferred to 6.4 ml of fresh OptiMEM.

The siRNA-oligofetamine mixture was then overlaid drop-wise on to cells and incubated for 4 hours, before addition of 4 ml of 30% FCS/OptiMEM. The medium was replaced by complete EBM with or without adenovirus 24 hours later.

### Migration Assay

Cell migration was measured by transwell assay. Transwells (pore size 8 μm, Becton Dickenson) were placed in 24 well plates containing 750 μl basal EBM in the presence or absence of factors. 2.5×10⁵ cells were trypisinised and neutralized with trypsine neutralising solution and were suspended on top of the transwell in 500 μl volume of basal EBM with 0.1% BSA. Cells were allowed to migrate at 37° C. for 4 hours. Non-migrated cells on the top surface of the transwell membrane were removed by a cotton swab. Cells that had migrated through pores and attached to the bottom side of the membranes, were stained with Reastain Quick-Diff Kit according to the manufacturer's instructions. Cells from four fields of equal size in the centre of the PET membrane were counted at ×100 magnification using an eyepiece indexed graticule. Each treatment was performed in duplicate.

### Oligonucleotide Microarray

Cells were treated with or without Ad-Egr3 or Ad-lacZ for 48 h. 5 μg of total RNA was extracted from cells using TRIzon reagent (Invitrogen) and purified with an RNeasy kit (Qiagen). RNA was converted to biotin labelled cRNA using the MessageAmp II-Biotin Amplification Kit (Affymetrix) as instructed and hybridised to the Affymetrix Human U133 2.0 array according to the manufacturer's instructions. GeneChips were washed and stained following protocols from Affymetrix and scanned on the GeneChip® Scanner 3000 7G (Affymetrix). Data was collected by using the GeneChip Operating Software (GCOS) and scaled to 100 (GCOS) prior to normalisation and differential gene expression was analysed using Genespring v7.3 (Agilent, USA). A gene was considered to be differentially expressed when the mean relative value for the gene was changed more than 2 fold in both comparisons, ie Egr3 vs untransfected, and Egr3 vs lacZ.

### Reverse Transcription and Real-Time PCR

Total RNA was extracted by using RNeasy Kit (Qiagen) and treated with DNAse I (Qiagen) for 15 min at room temperature to remove DNA. Single strand cDNA was synthesised from 2 μg of total RNA with oligo (dT)₁₂₋₁₈ primer in a 20 μl reaction volume using the SuperScript First-Strand Synthesis System for RT-PCR (Invitrogen). Primer pairs were designed using Prime3 software (frodo.wi.mit.edu/cgi-bin/primer3/primer3_www.cgi) and synthesised by Sigma Genosys. Real-time PCR was performed using the LightCycler-FastStart DNA Master SYBR Green I Kit and Lightcycler System (Roche Diagnostics) according to the manufacturer's protocol. PCR was conducted in duplicate for each sample and RNA preparations from at least three independent experiments were used for real-time RT-PCR. Data were normalised to the reference gene glyceraldehyde 3-phosphate dehydrogenase (GAPDH), and are presented as the mean fold change compared with LacZ control ±SEM.

### Immunoblotting

Cells were lysed with Laemmli buffer (2% SDS, 10% glycerol, 80 mM Tris-HCl and 1% □-mecaptoenthanol). Equal quantities of protein were separated by 10% SDS-polyacrylamide gel electrophoresis (SDS-PAGE) and electro-transferred to polyvinylidene difluoride membranes (Millipore). The membranes were blocked with PBS/3% BSA for 1 hour with agitation at room temperature and then incubated with primary antibody at 4° C. overnight. Membranes were washed in PBS/0.1% Tween 20 and then incubated with horseradish peroxidase (HRP)-conjugated secondary antibody (Dako) for 1 hour at room temperature. Immunoreactive bands were detected by chemiluminescence using ECL plus reagents (Amersham).

Activation of c-Met Receptor

Supernatants from Ad-Egr3 infected cells were collected as described above. Cells were incubated with cell supernatants and HGF 25 ng/ml as positive control respectively for 10 min. Cell lysates were prepared according to manufacturer's instruction. The phosphorylation of c-Met receptor was quantitatively determined using a sensitive and specific sandwich ELISA kits (PathScan Phospho®Met tyrosine 1234/1235) from Cell Signaling Technolgy. Levels of phosphorylated c-Met was normalised with respect to total c-Met level (PathScan Total met Sandwich ELISA Kit). Data was expressed as fold activation, scaled to untreated cells.

### ELISA

Cell culture supernatant conditioned medium were collected as described above. Level of growth factors and cytokines were measured using specific ELISA kits (R & D Systems), according to manufacturer's instructions.

### Preparation of Nuclear Extract and Egr DNA Binding Assay

Nuclear extracts were prepared using a Nuclear Extraction Kit (Active Motif, Rixensart, Belgium) according to the manufacturer's instructions. Briefly, cells treated as indicated, were collected by scraping, and pelleted by centrifugation at 500 rpm for 5 min at 4° C. The cell pellet was resuspended in the hypotonic buffer and incubated on ice for 15 min. Nuclei were pelleted by centrifugation at 13000 rpm for 30 sec. The supernatant (cytoplasmic fraction) was removed and the nuclei were resuspended in complete lysis buffer and placed on a rocking platform at 150 rpm at 4° C. for 30 min. The nuclear extract was centrifuged at 13000 rpm for 10 min at 4° C. and the supernatant fractions were frozen in aliquots at −80° C. after protein concentration was measured using Bio-Rad protein assay kit. Examination of Egr DNA binding was performed by a NoShift II transcription factor assay (Novagen) according to the manufacturer's instructions. Briefly, equal amounts of nuclear extracts were incubated for 20 min at room temperature with binding solution containing biotin-labelled consensus EGR response element (ERE) oligonucleotides following by further incubation for 20 min at room temperature in nuclease digestion buffer to remove the unbound oligonucleotides. The samples were then transferred into a 96 well capture plate containing oligos complementary to the tail of Egr consensus oligos. The samples were hybridised into the capture plate at room temperature for 45 min and were then incubated with detection solution containing streptavidin-alkaline phosphatase for 30 min at room temperature. The samples were incubated with chemiluminescent substrate at 37° C. for 30 min prior to measurement in a microplate luminometer. The Egr DNA binding activity was expressed as fold increase over the lacZ control.

### Time-Laps Microscopy

Cells were infected with Ad-Egr3 for 24 h. One millilitre of single cell suspension at concentration of 20,000 cells/ml in 2% FCS/basalEBM containing 20 mM of HEPES buffer were seeded on fibronectin coated 12 well plate at concentration 10 μg/ml for 4 hours before transferring to microscope chamber for video microscopy using a time-lapse interval of 10 min over 20 hours. To analyse cell spread area and elongation, images acquired by video microscopy were analysed with LaserPix software (Bio-Rad). Cell elongation was calculated as described by Dunn and Brown (1986) and was proportion of the long axis of cell to the shortest axis of the cells. Data are presented as mean+/−SEM of three independent experiments.

### Results

### Overexpression of Egr3 Enhances Egr3 DNA Binding and Induces NAB2 Expression

Adenoviral infection of Egr3 in HUVECs induced a time-dependent expression of Egr3 protein detected by both C-terminal and N-terminal antibodies. Egr3 protein, the 43 kDa band, started to increase after 9 h infection and reached a plateau after 15 hours. Increased expression of Ad-Egr3 protein persisted for 5 days. The expression of N-terminal truncated Egr3 was detected by C-terminal antibody as a 19 kDa band but was not detected by the N-terminal antibody.

A time course study of mRNA expression revealed that Ad-Egr3 gene expression started to increase 5 h after infection, preceding protein expression. There was an increase in Egr DNA binding activity in both Ad-Egr3 and Ad-ΔEgr3 infected cells, as expected since ΔEgr3 was deleted in transactivation domains but retained the DNA binding domain. The increase in Egr DNA binding began 24 h after Ad-Egr3 infection. In contrast, the expression of the known Egr3 target gene NAB2 started to increase 24 h after Ad-Egr3 infection, but was not affected by Ad-ΔEgr3 infection, suggesting that ΔEgr3 did not increase transcriptional activity, although it binds to EGRRE. These findings indicated that Ad-Egr3 infection increased Egr3 expression and transcriptional activity in endothelial cells.

### Overexpression of Egr3 Increases Cell Migration in Endothelial Cells

Since the Ad-Egr3 markedly enhanced Egr3 binding to its response element and induced downstream target gene expression, we then investigated the functions of Ad-Egr3 in endothelial cells. All the functional studies were performed 48 h after infection when the transcriptional activity reached its maximum. The Ad-Egr3 and Ad-ΔEgr3 infected cells exhibited no significant changes in cell proliferation and survival shown as in supplement materials. However, Ad-Egr3-infected cells demonstrated a striking increase in cell migration in a viral dosage dependent manner, whereas Ad-ΔEgr3 infected cells did not induced cell migration, indicating that overexpression of wild type Egr3 increased endothelial cell motility. Addition of VEGF to the lower wells of transwell plates caused a further increase in migration of Ad-Egr3-infected cells at all viral doses.

### Conditioned Medium of Ad-Egr3-Infected Cells Induces Chemotactic Activity

To investigate whether Ad-Egr3-infected cells produced secreted factors or factors that could stimulate increase cell migration, supernatant conditioned medium from Ad-Egr3- and Ad-lacZ-infected cells as well as uninfected cells, were collected and concentrated and then tested in the transwell migration assay. There was a significant increase in cell migration towards conditioned medium from Ad-Egr3-infected cells, compared with conditioned media from uninfected cells and Ad-lacZ infected cells, indicating that Ad-Egr3-infected cells increased chemotactic activity.

### Ad-Egr3 Upregulates Chemotactic Factors and Other Gene Expression

In order to understand the underlying mechanisms of Egr3 induced cell migration, RNAs from cells infected with Ad-Egr3, Ad-lacZ and uninfected HUVECs were extracted and gene expression was examined by Affymetrix oligonucleotide microarray analysis. Overexpression of Egr3 in HUVECs induced and downregulated multiple genes. These genes included genes involved in cell differentiation, morphogenesis, chemotaxis and migration, cell adhesion, cell signalling, sensory perception in nervous system development, metabolism, transcriptional regulation and ion transport. Selected genes were verified by quantitative real time RT-PCR. Increase expression of these factors HGF, CCL1, CX3CL1, CXCL14, CCL25 was confirmed by Q-PCR. However there were differential results on VEGF gene expression examined by Q-PCR. A 16 fold increase in VEGF expression was confirmed by a primer pairs detecting all isoforms of VEGF, whereas only 2.5 fold increase was identified by a primer pairs for exon 6 which only exists in secreted forms 165 and 121. Gene expression of matrix protein collagen type 1 α1 and laminin α1 was also increased. Interestingly Ad-Egr3 infection also induced gene expression of the haematopoietic stem cell markers CD52 and Thy1 and a gene highly expressed by endothelial progenitor cells APOE, whereas a known marker of mature endothelial cells, CD31, was strongly down-regulated.

Ad-Egr3 Induces HGF Protein Release and c-Met Receptor Activation

The results of oligonucleotide microarray and Q-PCR analysis raised the possibility that increased Egr3 expression might stimulate endothelial cell migration through increased autocrine production of one or more chemotactic factors. To determine which chemoattractant was mediating Ad-Egr3 infected cell migration, we first examined secreted proteins in conditioned medium of Ad-Egr3-infected cells by ELISA. CCL1, CX3CL1, and HGF proteins were detected in conditioned medium of Ad-Egr3-infected cells. HGF protein was 12 fold higher in conditional medium from Ad-Egr3 infected cells than from Ad-lacZ infected cells and rapidly increased 24 h after infection concomitantly with increase in Egr3 DNA binding, indicating that HGF expression could result from increased Egr3 transcriptional activity. This consequently raised another question whether Ad-Egr3 induced HGF had biological activity to activate its receptor. The conditional medium form Ad-Egr3 infected cells also stimulated c-Met receptor phosphorylation in uninfected cells with 1.8 fold increased on tyrosine 1234/1235 determined by PathScan Phospho-Met Sandwich ELISA in comparison with cell lysates from Ad-lacZ infected cells.

### HGF Mediates Endothelial Migration, and Inhibition of HGF Expression Suppresses Ad-Egr3 Mediated Endothelial Migration

We then tested the ability of CCL1, CCL25, CXCL14, CX₃CL1, and HGF as chemoattractants in cell migration by addition of recombinant proteins in the bottom well of transwell gradient assays. There was no significant increase in migration in response to CCL1, CCL25, CXCL14 and CX3CL1. In contrast, there was a dose dependent increase in cell migration towards HGF with a similar effect to that of VEGF.

To determine the effect of Ad-Egr3 induced HGF on chemotactic activity, cells were transfected with HGF specific siRNA 24 h before Ad-Egr3 infection in order to inhibit expression of synthesised HGF mRNA induced by Ad-Egr3 infection. HGF siRNA caused a striking inhibition of HGF mRNA compared with the non-targeted control siRNA 24 h after Ad-Egr3 infection. These results indicated that HGF siRNA was effective in preventing Ad-Egr3 induced HGF expression. To determine whether inhibition of HGF gene expression suppressed release of HGF protein, supernatants from HGF siRNA transfected cells followed by Ad-Egr3 infection were collected and examined HGF protein expression by ELISA. There was a striking reduction of HGF production in HGF siRNA and Ad-Egr3 treated cells in comparison with negative control siRNA and Ad-Egr3 treated cells. Furthermore, The same batches of supernatants were used as chemoattractants in migration assay, there was a significant reduction of chemotaxis towards the conditioned medium collected from Ad-Egr3-infected cells with HGF siRNA compared with negative control siRNA, indicating that Egr3 induced cell migration occurred through the upregulation and autocrine production of HGF.

### Ad-Egr3 Infection Induces Changes in Endothelial Shape and Polarity

Cells cultured on uncoated and on fibronectin coated plate at low density demonstrated a decreased cell spreading area, became elongated and acquired mesenchymal morphology, he movement of uninfected cells and Ad-lacZ infected cells mainly displayed a typical endothelial movement with protrusion of lamepodium at front with no preferential directions followed by contracting the rest of cell body and finally detracting from the rear. For next movement cells started protrusion randomly and repeated the same process. In contrast, Ad-Egr3 infected cells started with protrusion of filopodium at both ends. The movement is forward and backward in parallelled with cell long axis. Cells were looked more polarised than uninfected and Ad-IacZ infected cells.

These results clearly indicate that Ad-Egr3 infection induced endothelial chemotaxis is via the autocirne production of HGF. Since a truncated Ad-□Egr3, with a deletion of both transactivation domains, did not induce NAB2 gene expression and cell migration although it was able to increase Egr-DNA binding, it strongly suggests that Egr3 mediated gene expression and cell migration are mediated via Egr3 specific transcriptional activation.

Over expression of Egr3 not only induced production of chemotactic factors in endothelial cells, but also enhanced intrinsic motility of endothelial cells since there was an increase in cell migration towards basal EBM. Ad-Egr3 infected cells became elongated and polarised, and the morphology was changed from typical cobblestone shape to spindle shape and looked like mesencyhmal cells. However the gene array data on Ad-Egr3 infected cells did not reveal any increase in mesenchymal cell markers such as vimentin, CD29 and CD105.

The induction of several genes known as markers for haematopoietic cells and endothelial progenitor cells including Thy-1, CD52, and APOE and downregulation of endothelial marker CD31 suggests that overexpression of Egr3 is able to modulate differentiation status of endothelial cells to a phenotype with more progenitor-like characteristics. High levels of Egr3 have been found in several regions of central nerve system and other tissues during embryonic development.

1. A nucleotide which encodes a protein, wherein the protein includes an amino acid sequence having at least 95% homology to SEQ ID NO: 1 (Egr3 DNA binding domain), for use in therapy.

2. A nucleotide according to claim 1, wherein the protein includes an amino acid sequence having at least 95% homology to SEQ ID NO: 5 (ΔEgr3).

3. A nucleotide according to claim 1 or claim 2, wherein the protein includes an amino acid sequence having at least 95% homology to SEQ ID NO: 4 (Egr3).

4. A nucleotide according to any preceding claim, wherein the homology is 100%.

5. A vector comprising a nucleotide according to any preceding claim, for use in therapy.

6. A vector according to claim 5, which is an adenovirus.

7. A protein as defined in any of claims 1 to 4, for use in therapy.

8. A nucleotide, protein or vector according to any preceding claim, wherein the therapy is of a condition where neuronal regeneration is beneficial.

9. A nucleotide, protein or vector according to claim 8, wherein the condition is peripheral neuropathy, nerve injury, spinal injury or stroke.

10. A nucleotide, protein or vector according to any of claims 1 to 7, wherein the therapy is of a neovascular disease.

11. A nucleotide, protein or vector according to claim 10, wherein the neovascular disease is cancer, diabetic retinopathy, atherosclerosis or rheumatoid arthritis.
